(19) United States
(12) Patent Application Publication
(10) Pub. No.: US 2019/0224112 A1
(43) Pub. Date: Jul. 25, 2019

(54) Methods and Kit for Treating Skin Disorders

(71) Applicant: Lupin Atlantis Holdings SA, Zug (CH)

(72) Inventors: Matthew William Davis, Malvern, PA (US); Richard J. Holl, Baltimore, MD (US); Avinash Nangia, Sharon, MA (US); Vineeth Raghavan, Pune (IN); Amol Subhash Mandhare, Raleigh, NC (US); Mukesh Kumar, Pune (IN)

(21) Appl. No.: 15/879,958

(22) Filed: Jan. 25, 2018

(51) Int. Cl. A61K 9/00; A61K 31/573; A61K 9/107

(52) U.S. Cl. CPC A61K 9/0014; A61K 9/122; A61K 9/107; A61K 31/573

(57) ABSTRACT

A kit including topical pharmaceutical composition of corticosteroid(s) and metered-dose dispenser with an actuator which delivers precise amount of corticosteroid(s) per actuation for the treatment of various skin disorders is disclosed. Also disclosed is a process for preparation of such compositions.

### FIELD OF THE INVENTION

The present invention relates to a kit comprising topical pharmaceutical composition of therapeutic agent(s) and metered-dose dispenser and process for its preparation. More specifically, the present invention relates to a kit comprising liquid or semi-solid topical pharmaceutical composition comprising corticosteroid and metered-dose dispenser with an actuator for reliable administration of pharmaceutical composition. The present invention further relates to method of treating various skin disorders by administering pharmaceutical composition of the invention in metered-dose dispenser to patients in need thereof.

### BACKGROUND OF THE INVENTION

Topical drug delivery systems are preferred solution for treating various skin disorders locally. Such topical dosage forms include lotion, ointment, cream, gel, spray, powder, foam, solution, etc., which deliver the therapeutic agent(s) to diseased area of the skin.

Skin disorders occur worldwide in people of all races, genders and ages. These disorders are characterized by inflammation, irritation, redness, itching, flaking, plaque-type growth and scaling of the skin. Such skin disorders include, but are not limited to, psoriasis, dermatoses, eczema, lichenplanus, and seborrheic dermatitis (dandruff), discoid lupus erythematosus and other skin conditions related thereto.

Psoriasis and dermatitis are one of such inflammatory skin disorders, which exhibit itchiness, rash and red scaly patches on the skin. It generally affects various parts of the body like elbows, knees and scalp. Many therapeutic choices are available to treat these conditions, which include but not limited to topical drug delivery systems, phototherapy and systemic therapy. Generally, topical corticosteroids are most preferred choice of treatment for treating such skin conditions. Phototherapy and systemic therapy are generally considered secondary and used only when topical drug delivery systems containing corticosteroids fail in treating it.

Corticosteroids are classified based on their potency in the vasoconstrictor assay (VCA), also called as skin blanching assay. Accordingly, corticosteroids can be classified as super potent (Class 1), high potent (Class 2), upper mid strength (Class 3), mid strength (Class 4), lower mid strength (Class 5), low potent (Class 6) and least potent (Class 7). Preparations containing super potent (Class I) corticosteroids, are most efficacious but also exhibit greater side effects.

Prolonged use of high doses of such topical corticosteroids, or treatment of large areas of skin, can cause hypercorticism or suppression of hypothalamic-pituitary-adrenal (HPA) axis. As a result, administration of potent class topical corticosteroids is restricted to set amounts dependent on the specific corticosteroid. Potent corticosteroids are therefore generally prescribed for a short duration of time in small amounts and, as such, typically used without any pronounced side effects. When it is necessary to administer these drugs for longer therapeutic periods, accurate dosing is of paramount importance to avoid serious side effects, especially with super potent (Class I) corticosteroids. Therefore dosages to be administered for such corticosteroids to the patient are restricted by Food and Drug Administration (FDA) to avoid over-dosage related side effects.

Conventionally, topical corticosteroids are available as aerosol, foam, gel, cream, lotion and ointment and are also disclosed in various prior arts, incorporated herein as reference only. U.S. Pat. No. 3,892,856 A describes compositions comprising corticosteroids dissolved in polyethylene glycol and emulsified into an oleaginous base. U.S. Pat. No. 3,934,013 A describes topical pharmaceutical compositions containing two corticosteroids, propylene glycol, fatty alcohol and water. U.S. Pat. No. 4,343,798 A discloses topical antimicrobial/anti-inflammatory compositions containing fatty acids and corticosteroids. WO 2011026076 A1 discloses topical sprayable compositions comprising steroid as an active agent. U.S. Pat. No. 7,078,058 B2 discloses betamethasone valerate aerosols with a quick-break foaming agent, ethanol, propellant and buffering agent.

It has been observed that, currently available corticosteroids formulations have certain disadvantages. For example, Clobex® (clobetasol propionate) spray has a larger coverage area and may result in spray droplets entering the nasal cavity or eyes. Clobex® (clobetasol propionate) lotion is packaged in high-density polyethylene squeeze bottles with a disc-top closure. As such, this packaging configuration provides no means of accurately and reliably dispensing and/or administering the lotion to affected skin.

Hence, there is still an unmet need for subject compliant topical formulations that are effective in the treatment of skin disorders, and which can accurately administer precise dosage(s) of pharmaceutical composition at desired site of action, with decreased inconvenience and irritation, increased ease of use for the subject, longer duration of action and also reduced risk of systemic side effects due to exposure to potent corticosteroid(s).

### SUMMARY OF THE INVENTION

Principal objective of the present invention is to formulate a kit comprising stable topical pharmaceutical composition comprising corticosteroid(s) and metered-dose dispenser.

Another objective of the invention is to formulate a kit comprising stable liquid or semi-solid topical pharmaceutical composition comprising corticosteroid(s) or its pharmaceutically acceptable derivatives, including but not limited to addition salts, hydrates, solvates, ethers, esters and mixtures thereof and metered-dose dispenser with an actuator for reliable administration of pharmaceutical composition.

Yet another objective of the invention is to develop a process for preparation of kit comprising efficacious and stable liquid or semi-solid compositions comprising corticosteroid(s) or its pharmaceutically acceptable derivatives, including but not limited to addition salts, hydrates, solvates, ethers, esters and mixtures thereof and metered-dose dispenser with an actuator for reliable administration of pharmaceutical composition.

Further objective of the invention is to treat various skin disorders by using the kit comprising pharmaceutical composition of the invention and metered-dose dispenser with an actuator for reliable administration of pharmaceutical composition to patients in need thereof.

The present invention features a kit comprising liquid or semi-solid topical pharmaceutical composition comprising corticosteroid(s) or its pharmaceutically acceptable derivatives, including but not limited to addition salts, hydrates, solvates, ethers, esters and mixtures thereof and metered-dose dispenser with an actuator for accurate administration of pharmaceutical composition.

In one embodiment of the present invention, corticosteroid is clobetasol or its pharmaceutically acceptable derivatives, including but not limited to addition salts, hydrates, solvates, ethers, esters and mixtures thereof.

In further embodiment of the present invention, pharmaceutical composition is selected from topical dosage forms like cream, ointment, gel, lotion and solution, biphasic or multiphasic emulsion, suspension, aerosol, spray, foam, shampoo, etc.

In one embodiment of the present invention, actuator is selected from mechanical or electromechanical system. Actuator may be attached to metered-dose dispenser or provided separately.

In another embodiment of the present invention, metered-dose dispenser comprises dose counter mechanism selected from physical, electronic, electro-mechanical, or mechanical systems.

In further embodiment of the present invention, metered-dose dispenser comprises a cap, which may be used as an applicator for pharmaceutical composition of the present invention.

In one embodiment, present invention provides method of treating various skin disorders by topically administering and monitoring number of actuations of liquid or semi-solid pharmaceutical composition of the invention to a patient in need thereof.

In another embodiment, skin disorder is corticosteroid-responsive dermatoses. In yet another embodiment, skin condition is moderate to severe plaque psoriasis.

In further embodiment, present invention reduces the adverse side effects by accurately monitoring and optimizing the dosage amounts of corticosteroids.

### BRIEF DESCRIPTION OF THE DRAWINGS

The present invention is further described with references to the following drawings, wherein:

FIG. 1 shows a metered-dose dispenser with over-cap;

FIG. 2a shows an actuator with cap;

FIG. 2b shows a different view of the actuator with cap;

FIG. 3 shows a bottle component of the metered-dose dispenser.

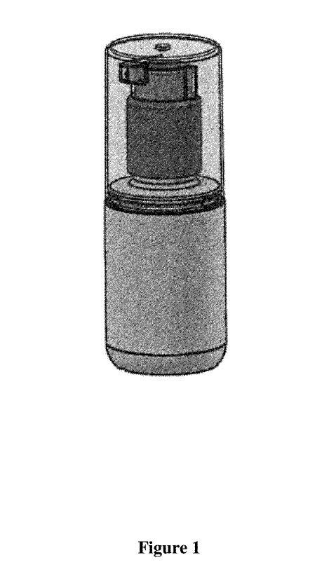

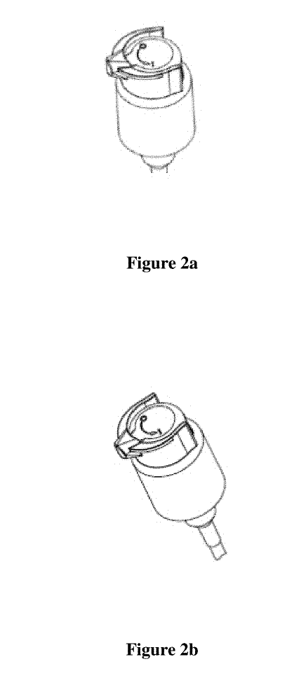

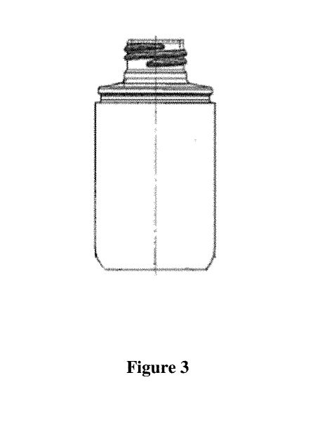

### DETAILED DESCRIPTION OF THE INVENTION

The present invention relates to a kit comprising liquid or semi-solid topical pharmaceutical composition comprising corticosteroid(s) or its pharmaceutically acceptable derivatives, including but not limited to addition salts, hydrates, solvates, ethers, esters and mixtures thereof and metered-dose dispenser with an actuator for reliable administration of pharmaceutical composition, specifically useful for dispensing accurate dosage of pharmaceutically acceptable composition of the therapeutic agent or combinations thereof.

The present invention also provides method of treating various skin disorders by topically administering and monitoring the number of actuations of liquid or semi-solid pharmaceutical composition of the invention to patient in need thereof.

As used herein, the term “therapeutic agent” refers to skin modulating agents such as retinoic acid, retinol, retinal, retinoids and esters thereof; vitamin D and derivatives thereof; estrogens such as estradiol, kojic acid or hydroquinone; antibacterial agents such as clindamycin phosphate, erythromycin or tetracycline class of antibiotics; antiparasitic agents such as metronidazol, crotamiton or pyrethrinoids; antifungal agents such as econazole, ketoconazole or miconazole or salts thereof; polyene compounds such as amphotericin B; allylamines compounds such as terbinafine or alternatively octopirox; steroidal anti-inflammatory agents such as super potent topical corticosteroids, for example clobetasol propionate, diflorasone diacetate, high-potency topical corticosteroids such as betamethasone valerate, betamethasone diproprionate, diflucortolone valerate, clobetasol butyrate, fluticasone valerate, hydrocortisone 17-butyrate, mometasone furoate, halobetasol propionate, desoximetasone, flucinonide; non-steroidal anti-inflammatory agents such as ibuprofen, diclofenac, acetylsalicylic acid, acetaminophen or glycyrrhetinic acid and salts thereof; anesthetic agents such as lidocaine hydrochloride and derivatives thereof; antipruritic agents such as thenaldine, trimeprazine or cyproheptadine; antiviral agents such as acyclovir; keratolytic agents such as alpha- and beta-hydroxycarboxylic or beta-ketocarboxylic acids, their salts, amides or esters and more particularly hydroxy acids such as glycolic acid, lactic acid, malic acid, salicylic acid, citric acid and, in general, the fruit acids, and 5-N-octanoylsalicylic acid; anti-free radical agents such as alpha-tocopherol or esters thereof, superoxide dismutases, certain metal chelators or ascorbic acid and esters thereof; antiseborrhoeic agents such as progesterone; antidandruff agents such as octopirox or zinc pyrithione; anti-acne agents such as retinoic acid, benzoyl peroxide or adapalene; antimetabolites; agents for combating hair loss such as minoxidil; zinc pyrithione, tar compounds like coal tar, juniper tar, birch tar, pine tar, vegetable tar or mineral tar; anti-psoriatic agents such as allantoin, menthol, phenol, undecylenate compounds, spermidine, spermine, putrescine, 5-amino or substituted amino 1,2,3-triazoles, halomethyl derivatives of alpha-amino acids, phenyl alpha-acyloxyacetamides, resorcin, eosin; and combinations thereof.

Compositions of the present invention comprise from about 0.0001% to about 20% by weight, preferably from about 0.025% to about 15% by weight, relative to total weight of the composition comprising at least one therapeutic agent.

As used herein, the term “pharmaceutically acceptable composition” refers to liquid or semi-solid dosage form(s) such as dispersions, suspensions, creams, ointments, foams, aerosols, lotions, solutions, emulsions, micro emulsions, sprays, gels and the like, solid dispersion, injection preparations, lyophilized formulations, modified release formulations, delayed release formulations, extended release formulations, pulsatile release formulations, dual release formulations and the like may also be envisaged under the ambit of present invention.

As used herein, the term “metered-dose dispenser” refers to a device which is assembled from different components including actuator, cap, over-cap, bottle and dose counter.

As used herein, the term “actuator” refers to a component of metered-dose dispenser, which releases single metered dose of pharmaceutically acceptable composition containing therapeutic ingredient(s). The metered-dose dispenser delivers pharmaceutically acceptable composition upon actuation, optionally after priming. The number of priming actuations may vary between 0 to 30. The most preferable range for priming is 1 to 10.

Metered-dose dispenser of present invention delivers about 0.10 to about 1.0 gm of the pharmaceutically acceptable composition per actuation. More preferably, metered-dose dispenser of the present invention delivers about 0.10 to about 0.5 gm of pharmaceutically acceptable composition per actuation. Most preferably, metered-dose dispenser of the present invention delivers about 0.15 to about 0.3 gm of the pharmaceutically acceptable composition per actuation.

As used herein, the term “about” refers to a numeric value, including, for example, whole numbers, fractions, and percentages, whether or not explicitly indicated. The term “about” generally refers to a range of numerical values (e.g., ±10-30% of the recited value) that one of ordinary skill in the art would consider equivalent to the recited value (e.g., having the same function or result). In some instances, the term “about” may include numerical values that are rounded to nearest significant figure and may cover variation of ±100-200%.

In one aspect of the present invention, metered-dose dispenser comprises a dose counter for recording number of actuations, wherein said metered-dose dispenser allows patient to more effectively monitor the dosing so as not to exceed maximum recommended dose within a specified period of time.

The term “dose counter” includes both mechanisms that may use numeric count to indicate doses remaining, as well as dose-indicating mechanisms that do not enumerate the number of actuations, but rather indicate via color coding or other means when a metered-dose dispenser is nearing the end of its useful life. In another aspect of the present invention, metered-dose dispenser further comprises dose counter mechanism selected from physical, electronic, electro-mechanical, or mechanical systems. In one embodiment, dose counter may be attached with an actuator of the metered-dose dispenser or actuator can itself act as a dose counter. In another embodiment, dose counter may be provided with kit or may be attached with metered-dose dispenser.

In other aspect of the present invention, viscosity of pharmaceutical composition which significantly affects performance of metered-dose dispenser is about 0.01 to 30 poise. More preferably, viscosity of pharmaceutically acceptable composition of the present invention is about 0.1 to 10 poise. Most preferably, viscosity of pharmaceutically acceptable composition is about 0.5 to 5 poise.

In other aspect of the present invention, bottle of metered-dose dispenser is constructed from high density polyethylene (HDPE) and/or other regulatory acceptable materials of construction. Bottle can also be constructed from the material selected from polycarbonate, polyethylene terephthalate, polyethylene terephthalate glycol-modified, polypropylene, and silicone-based materials or combinations thereof. Volume capacity of the bottle varies based on the requirement. Most preferable volume of the bottle is about 30 ml to about 200 ml.

In another aspect of the present invention, metered-dose dispenser has integral pump locking mechanism which provides child resistant feature to avoid use of potent corticosteroids by children. In one of the embodiments, actuator contains lock/unlock symbols in various pictorial forms like open/unopened locks, open/closed flowers to increase patients or caregivers ease of use and compliance.

In another aspect of the present invention, pharmaceutically acceptable composition is released through an actuator having a tip, such that any leftover composition at the tip of actuator is sucked back after each actuation into bottle to avoid spillover and unintentional contact with body parts of the patient.

Metered-dose dispenser of the present invention comprises a cap and over-cap. Cap of metered-dose dispenser can be further modified to use as an applicator for application of pharmaceutically acceptable composition at the site of action, further serving complete transfer of composition at desired site and reducing the chances of over-dose or application at unintentional body parts. Such use of cap provides “no touch” treatment option to the patient or caregiver, which further improves patient compliance. The over-cap prevents contamination to metered-dose dispenser.

Dosing regimen for proposed product of the present invention which is to be administered from metered-dose dispenser will depend on the surface area of skin to be treated. Published literature suggests use of fingertip unit (FTU) as a common practice for estimating amount of product needed. As per Long and Finlay's article published in ‘Clinical and Experimental Dermatology’ in 1991, one FTU is approximately 0.5 gm of lotion and can cover approximately 2 palm areas; 1 palm area is approximately 0.8% of Body Surface Area (BSA). Accordingly, as one of the embodiments as per present invention delivers about 0.30±20% gm of lotion per actuation, it covers about 1.2 palm areas which in turn would correspond to approximately 1% BSA.

Present invention also provides control mechanism for dosage form administration through metered dose dispenser by controlling the exposure of potent corticosteroid to safest recommended levels by FDA i.e. amount of potent corticosteroid medication to be delivered to patient's skin surface should be strictly not more than 50 gm per week. Inventors of the present invention optimized the number of actuations per administration of metered dose dispenser, which will give safe, reliable and effective method of administration of potent corticosteroid(s) using the kit of present invention. Inventors have established the correlation between dispensed weight and number of actuations needed per administration to control the amount to be dispensed to 50 gm per week. Following table gives the optimized dosing matrix for one of the embodiments of the invention to facilitate this correlation.

TABLE 1
Dosing Matrix

| | | | | | | | Quantity |
|---|---|---|---|---|---|---|---|
| | | | | | | | of |
| | Delivered | | Skin | | Total | | Cortico- |
| | Cortico- | Lotion | surface | | Cortico- | Total | steroid |
| | steroid | dispensed | area | Total | steroid | lotion | lotion |
| Actuations | dose per | weight per | per | actua- | per | per | per |
| per | admini- | admini- | admini- | tions | day, | day, | week, |
| admini- | stration, | stration, | stration, | per | mg | gm | gm |
| stration | mg (±20%) | gm (±20%) | cm² (±20%) | day | (±20%) | (±20%) | (±20%) |
| 1 | 0.150 | 0.300 | 181.5 | 2 | 0.300 | 0.600 | 4.2 |
| 2 | 0.300 | 0.600 | 363 | 4 | 0.600 | 1.200 | 8.4 |
| 3 | 0.450 | 0.900 | 544.5 | 6 | 0.900 | 1.800 | 12.6 |
| 4 | 0.600 | 1.200 | 726 | 8 | 1.200 | 2.400 | 16.8 |
| 5 | 0.750 | 1.500 | 907.5 | 10 | 1.500 | 3.000 | 21 |
| 6 | 0.900 | 1.800 | 1089 | 12 | 1.800 | 3.600 | 25.2 |
| 7 | 1.050 | 2.100 | 1270.5 | 14 | 2.100 | 4.200 | 29.4 |
| 8 | 1.200 | 2.400 | 1452 | 16 | 2.400 | 4.800 | 33.6 |
| 9 | 1.350 | 2.700 | 1633.5 | 18 | 2.700 | 5.400 | 37.8 |
| 10 | 1.500 | 3.000 | 1815 | 20 | 3.000 | 6.000 | 42 |
| 11 | 1.650 | 3.300 | 1996.5 | 22 | 3.300 | 6.600 | 46.2 |
| 12 | 1.800 | 3.600 | 2178 | 24 | 3.600 | 7.200 | 50.4 |

In another embodiment of the present invention, dosing matrix is optimized such that not more than 12 pumps per application and 24 pumps per day to be used so that total dosage will not exceed 50 gm per week.

In another aspect, present invention provides a method of treating skin disorder with precise dose administration at the affected site wherein precise dose is delivered by metered-dose dispenser. It is recommended that, treatment of inflammatory and pruritic manifestations of corticosteroid-responsive dermatoses should be limited to 2 consecutive weeks only and total dosage should not exceed 50 gm per week. For the treatment of moderate to severe plaque psoriasis, localized lesions (less than 10% body surface area) that have not sufficiently improved after the initial 2-week treatment, treatment could be continued for up to 2 additional weeks. Any additional benefits of extending treatment should be weighed against the risk of HPA axis suppression before prescribing for more than 2 weeks.

The foregoing description describes a kit comprising liquid or semi-solid topical pharmaceutical composition comprising corticosteroid(s) and metered-dose dispenser and its use in treating skin disorders using precise dose delivered by metered-dose dispenser, especially in corticosteroid responsive dermatoses and moderate to severe plaque psoriasis. It is to be understood that pharmaceutically acceptable composition(s) of the present invention and construction of various parts of the metered-dose dispenser can be modified to meet specific requirements.

The following examples are for purpose of illustration of the present invention only and are not intended in any way to limit the scope of present invention.

### Example 1: Metered-Dose Dispenser Comprising Clobetasol Propionate Lotion

| S. No. | Name of Ingredients | % w/w |
|---|---|---|
| 1 | Clobetasol Propionate | 0.05 |
| 2 | Hypromellose | 0.10 |
| 3 | Carbomer/Pemulen | 0.30 |
| 4 | Propylene glycol | 45.00 |
| 5 | Mineral oil | 18.00 |
| 6 | Polyoxyethylene glycol 300 | 1.00 |
| | isostearate | |
| 7 | Sodium hydroxide | q.s. to adjust pH |
| | | between 4.2 and 6.5 |
| 8 | Purified water | q.s. to 100 |

Procedure: Aqueous phase was prepared by dispersing hypromellose and carbomer in mixture of purified water and propylene glycol. Oil phase was prepared with mineral oil and polyoxyethylene glycol 300 isostearate. Further oil phase was added to aqueous phase and pH of the emulsified bulk was adjusted. Clobetasol propionate solution was then added to emulsified bulk and homogenized.

### Example 2: Metered-Dose Dispenser Comprising Betamethasone Dipropionate Lotion

| S. No. | Name of Ingredients | % w/w |
|---|---|---|
| 1 | Betamethasone Dipropionate | 0.10 |
| 2 | Hypromellose | 0.15 |
| 3 | Carbomer/Pemulen | 0.40 |
| 4 | Propylene glycol | 47.00 |
| 5 | Mineral oil | 20.00 |
| 6 | Polyoxyethylene glycol 300 isostearate | 1.50 |
| 7 | Sodium hydroxide | q.s. to adjust pH |
| | | between 4.2 and 6.5 |
| 8 | Purified water | q.s. to 100 |

Procedure: Aqueous phase was prepared by dispersing hypromellose and carbomer in mixture of purified water and propylene glycol. Oil phase was prepared with mineral oil and polyoxyethylene glycol 300 isostearate. Further oil phase was added to aqueous phase and pH of the emulsified bulk was adjusted. Betamethasone dipropionate solution was then added to emulsified bulk and homogenized.

### Example 3: Metered-Dose Dispenser Comprising Hydrocortisone Valerate Lotion

| S. No. | Name of Ingredients | % w/w |
|---|---|---|
| 1 | Hydrocortisone Valerate | 0.20 |
| 2 | Hypromellose | 0.20 |
| 3 | Carbomer/Pemulen | 0.50 |
| 4 | Propylene glycol | 50.00 |
| 5 | Mineral oil | 22.00 |
| 6 | Polyoxyethylene glycol 300 isostearate | 2.00 |
| 7 | Sodium hydroxide | q.s. to adjust pH |
| | | between 4.2 and 6.5 |
| 8 | Purified water | q.s. to 100 |

Procedure: Aqueous phase was prepared by dispersing hypromellose and carbomer in mixture of purified water and propylene glycol. Oil phase was prepared with mineral oil and polyoxyethylene glycol 300 isostearate. Further oil phase was added to aqueous phase and pH of the emulsified bulk was adjusted. Hydrocortisone valerate solution was then added to emulsified bulk and homogenized.

### Example 4: Metered-Dose Dispenser Comprising Hydrocortisone Butyrate Lotion

| S. No. | Name of Ingredients | % w/w |
|---|---|---|
| 1 | Hydrocortisone Butyrate | 0.10 |
| 2 | Hypromellose | 0.10 |
| 3 | Carbomer/Pemulen | 0.30 |
| 4 | Propylene glycol | 45.00 |
| 5 | Mineral oil | 18.00 |
| 6 | Polyoxyethylene glycol 300 isostearate | 1.00 |
| 7 | Sodium hydroxide | q.s. to adjust pH |
| | | between 4.2 and 6.5 |
| 8 | Purified water | q.s. to 100 |

Procedure: Aqueous phase was prepared by dispersing hypromellose and carbomer in mixture of purified water and propylene glycol and dispersing hydrocortisone butyrate under heating. Oil phase was prepared with mineral oil and polyoxyethylene glycol 300 isostearate. Further oil phase was added to aqueous phase and pH of the emulsified bulk was adjusted.

### Example 5: Metered-Dose Dispenser Comprising Methylprednisolone Acetate Lotion

| S. No. | Name of Ingredients | % w/w |
|---|---|---|
| 1 | Methylprednisolone Acetate | 0.05 |
| 2 | Hypromellose | 0.15 |
| 3 | Carbomer/Pemulen | 0.50 |
| 4 | Propylene glycol | 50.00 |
| 5 | Mineral oil | 22.00 |
| 6 | Polyoxyethylene glycol 300 isostearate | 2.00 |
| 7 | Sodium hydroxide | q.s. to adjust pH |
| | | between 4.2 and 6.5 |
| 8 | Purified water | q.s. to 100 |

Procedure: Aqueous phase was prepared by dispersing hypromellose and carbomer in mixture of purified water and propylene glycol. Oil phase was prepared with mineral oil and polyoxyethylene glycol 300 isostearate. Further oil phase was added to aqueous phase and pH of the emulsified bulk was adjusted. Methylprednisolone acetate solution was then added to emulsified bulk and homogenized.

### Example 6: Metered-Dose Dispenser Comprising Hydrocortisone Butyrate Lotion

| S. No. | Name of Ingredients | % w/w |
|---|---|---|
| 1 | Hydrocortisone Butyrate | 0.100 |
| 2 | Light Mineral Oil | 7.00 |
| 3 | Cetostearyl Alcohol | 2.50 |
| 4 | Ceteth-20 | 2.00 |
| 5 | White Petrolatum | 1.50 |
| 6 | Butylated Hydroxytoluene | 0.02 |
| 7 | Propylparaben | 0.10 |
| 8 | Butylparaben | 0.05 |
| 9 | Citric Acid | 0.25 |
| 10 | Sodium Citrate | 0.25 |
| 11 | Purified Water | q.s. to 100 |

Procedure: Aqueous phase was prepared by dissolving citric acid, sodium citrate and ceteth-20 in purified water and dispersing hydrocortisone butyrate under heating. Oil phase was prepared by mixing light mineral oil, cetostearyl alcohol, ceteth-20, white petrolatum, butylated hydroxytoluene, propyl paraben and butyl paraben under moderate heating. Further oil phase was added to aqueous phase under homogenization and stirring for emulsification and then cooled to room temperature.

### Example 7: Metered-Dose Dispenser Comprising Clobetasol Butyrate Lotion

| S. No. | Name of Ingredients | % w/w |
|---|---|---|
| 1 | Clobetasol Butyrate | 0.100 |
| 2 | Light Mineral Oil | 8.00 |
| 3 | Cetostearyl Alcohol | 3.50 |
| 4 | Ceteth-20 | 3.00 |
| 5 | White Petrolatum | 2.00 |
| 6 | Butylated Hydroxytoluene | 0.035 |
| 7 | Propylparaben | 0.20 |
| 8 | Butylparaben | 0.07 |
| 9 | Citric Acid | 0.45 |
| 10 | Sodium Citrate | 0.45 |
| 11 | Purified Water | q.s. to 100 |

Procedure: Aqueous phase was prepared by dissolving citric acid, sodium citrate and ceteth-20 in purified water. Oil phase was prepared by mixing light mineral oil, cetostearyl alcohol, ceteth-20, white petrolatum, Clobetasol butyrate, propyl paraben and butyl paraben under heating. Further oil phase was added to aqueous phase under homogenization and stirring for emulsification and then cooled to room temperature.

### Example 8: Metered-Dose Dispenser Comprising Halobetasol Propionate Lotion

| S. No. | Name of Ingredients | % w/w |
|---|---|---|
| 1 | Halobetasol Propionate | 0.05% |
| 2 | Light Mineral Oil | 8.50 |
| 3 | Cetostearyl Alcohol | 4.50 |
| 4 | Ceteth-20 | 4.00 |
| 5 | White Petrolatum | 2.50 |
| 6 | Butylated Hydroxytoluene | 0.05 |
| 7 | Propylparaben | 0.30 |
| 8 | Butylparaben | 0.10 |
| 9 | Citric Acid | 0.55 |
| 10 | Sodium Citrate | 0.60 |
| 11 | Purified Water | q.s. to 100 |

Procedure: Aqueous phase was prepared by dissolving citric acid, sodium citrate and ceteth-20 in purified water and dispersing Halobetasol propionate under heating. Oil phase was prepared by mixing light mineral oil, cetostearyl alcohol, ceteth-20, white petrolatum, propyl paraben and butyl paraben under moderate heating. Further oil phase was added to aqueous phase under homogenization and stirring for emulsification and then cooled to room temperature.

### Example 9: Metered-Dose Dispenser Comprising Clobetasol Propionate Cream

| S. No. | Name of Ingredients | % w/w |
|---|---|---|
| 1 | Clobetasol Propionate | 0.05 |
| 2 | Propylene glycol | 10.00 |
| 3 | Cetosteryl alcohol | 10.00 |
| 4 | Cetyl esters wax | 3.00 |
| 5 | Octyldodecanol | 10.00 |
| 6 | Sorbitan monosterate | 2.00 |
| 7 | Polysorbate | 1.00 |
| 8 | Triethanolamine | q.s. to adjust pH to 6 |
| | (Added, only if required) | |
| 9 | Purified water | q.s. to 100 |

Procedure: Aqueous phase was prepared by mixing propylene glycol and Clobetasol propionate and then purified water was added under heating. Oil phase was prepared by mixing cetostearyl alcohol, cetyl esters wax, octyldodecanol, sorbitan monostearate and polysorbate under heating. Further oil phase was added to aqueous phase under homogenization and stirring for emulsification and then cooled to room temperature. pH was Adjusted to 6 Using Triethanolamine, if required.

### Example 10: Metered-Dose Dispenser Comprising Clobetasol Propionate Cream

| S. No. | Name of Ingredients | % w/w |
|---|---|---|
| 1 | Clobetasol Propionate | 0.04 |
| 2 | Propylene glycol | 10.00 |
| 3 | White petrolatum | 6.50 |
| 4 | Isostearic acid | 5.00 |
| 5 | Cetyl alcohol | 3.00 |
| 6 | Stearyl alcohol | 4.00 |
| 7 | Sorbitan monosterate | 3.00 |
| 8 | Polysorbate | 0.60 |
| 9 | Triethanolamine | q.s. to adjust pH to 6 |
| | (Added, only if required) | |
| 10 | Xanthan gum | 0.5 |
| 11 | Purified water | q.s. to 100 |

Procedure: Aqueous phase was prepared by adding Clobetasol propionate in purified water and adding xanthan gum under heating. Oil phase was prepared by mixing white petrolatum, isostearic acid, cetyl alcohol, stearyl alcohol, sorbitan monostearate and polysorbate under heating. Further oil phase was added to aqueous phase under homogenization and stirring for emulsification and then cooled to room temperature. pH was adjusted to 6 using triethanolamine, if required.

### Example 11: Metered-Dose Dispenser Comprising Clobetasol Propionate Gel

| S. No. | Name of Ingredients | % w/w |
|---|---|---|
| 1 | Clobetasol Propionate | 0.03 |
| 2 | Carbomer | 0.50 |
| 3 | Alcohol | 12.00 |
| 4 | Propylene glycol | 20.00 |
| 5 | Butylated hydroxytoluene (BHT) | 0.10 |
| 6 | Triethanolamine | q.s. to adjust pH 5.5 to 6.5 |
| 7 | Purified water | q.s. to 100 |

Procedure: Carbomer was dispersed in a mixture of purified water and propylene glycol under stirring. Clobetasol propionate and butylated hydroxytoluene were dissolved in Alcohol and added to carbomer dispersion under stirring. pH of gel was adjusted using Triethanolamine.

### Example 12: Metered-Dose Dispenser Comprising Clobetasol Propionate Topical Solution

| S. No. | Name of Ingredients | % w/w |
|---|---|---|
| 1 | Clobetasol Propionate | 0.05 |
| 2 | Hydroxypropyl Cellulose | 2.00 |
| 3 | Alcohol | 25.00 |
| 4 | Propylene glycol | 12.00 |
| 5 | Purified Water | q.s. to 100 |

Procedure: Hydroxypropyl cellulose was dispersed in a mixture of purified water and propylene glycol under stirring. Clobetasol propionate was dissolved in alcohol and added to hydroxypropyl cellulose dispersion under stirring.

### Example 13: Metered-Dose Dispenser Comprising Clobetasol Propionate Gel

| S. No. | Name of Ingredients | % w/w |
|---|---|---|
| 1 | Clobetasol Propionate | 0.025 |
| 2 | Carbomer | 0.70 |
| 3 | Alcohol | 14.50 |
| 4 | Propylene glycol | 30.00 |
| 5 | Butylated hydroxytoluene (BHT) | 0.30 |
| 6 | Triethanolamine | q.s. to adjust pH 5.5 to 6.5 |
| 7 | Purified water | q.s. to 100 |

Procedure: Carbomer was dispersed in a mixture of purified water and propylene glycol under stirring. Clobetasol propionate and butylated hydroxytoluene were dissolved in Alcohol and added to carbomer dispersion under stirring. pH of gel was adjusted using Triethanolamine.

### Example 14: Metered-Dose Dispenser Comprising Clobetasol Propionate Topical Solution

| S. No. | Name of Ingredients | % w/w |
|---|---|---|
| 1 | Clobetasol Propionate | 0.025 |
| 2 | Hydroxypropyl Cellulose | 3.00 |
| 3 | Alcohol | 30.00 |
| 4 | Propylene glycol | 15.00 |
| 5 | Purified Water | q.s. to 100 |

Procedure: Hydroxypropyl cellulose was dispersed in a mixture of purified water and propylene glycol under stirring. Clobetasol propionate was dissolved in alcohol and added to hydroxypropyl cellulose dispersion under stirring.

### Example 15: Metered-Dose Dispenser Comprising Clobetasol Propionate Cream

| S. No. | Name of Ingredients | % w/w |
|---|---|---|
| 1 | Clobetasol Propionate | 0.05 |
| 2 | Propylene glycol | 15.00 |
| 3 | Cetosteryl alcohol | 12.00 |
| 4 | Cetyl esters wax | 4.50 |
| 5 | Octyldodecanol | 2.50 |
| 6 | Sorbitan monosterate | 3.00 |
| 7 | Polysorbate | 2.00 |
| 8 | Triethanolamine | q.s. to adjust pH to 6 |
| | (Added, only if required) | |
| 9 | Purified water | q.s. to 100 |

Procedure: Aqueous phase was prepared by mixing propylene glycol and Clobetasol propionate and then purified water was added under heating. Oil phase was prepared by mixing cetostearyl alcohol, cetyl esters wax, octyldodecanol, sorbitan monostearate and polysorbate under heating. Further oil phase was added to aqueous phase under homogenization and stirring for emulsification and then cooled to room temperature. pH was adjusted to 6 using triethanolamine, if required.

### Example 16: Metered-Dose Dispenser Comprising Clobetasol Propionate Cream

| S. No. | Name of Ingredients | % w/w |
|---|---|---|
| 1 | Clobetasol Propionate | 0.04 |
| 2 | Propylene glycol | 15.00 |
| 3 | White petrolatum | 8.50 |
| 4 | Isostearic acid | 8.50 |
| 5 | Cetyl alcohol | 5.00 |
| 6 | Stearyl alcohol | 6.00 |
| 7 | Sorbitan monosterate | 4.50 |
| 8 | Polysorbate | 2.00 |
| 9 | Triethanolamine | q.s. to adjust pH to 6 |
| | (Added, only if required) | |
| 10 | Xanthan gum | 0.5 |
| 11 | Purified water | q.s. to 100 |

Procedure: Aqueous phase was prepared by adding Clobetasol propionate in purified water and adding xanthan gum under heating. Oil phase was prepared by mixing white petrolatum, isostearic acid, cetyl alcohol, stearyl alcohol, sorbitan monostearate and polysorbate under heating. Further oil phase was added to aqueous phase under homogenization and stirring for emulsification and then cooled to room temperature. pH was adjusted to 6 using triethanolamine, if required.

Physicochemical characterization of prepared pharmaceutical composition(s) were carried out and were found to be within acceptable limits for appearance, texture, pH, viscosity and assay.

Metered dose dispenser with pharmaceutical composition(s) of the present invention were also evaluated for Delivered-Dose Uniformity over the entire container, Delivered-Dose Uniformity within different metered dose dispensers, Pump Performance Test (Number of metered pump actuations per metered dose dispenser) and were found to be passing the acceptance criteria in both inverted and up-right positions.

1. A kit comprising:
a. liquid or semi-solid topical pharmaceutical composition comprising clobetasol propionate, and
b. a metered-dose dispenser,
wherein the metered-dose dispenser delivers a precise amount of a lotion comprising clobetasol propionate per actuation, wherein the lotion has a viscosity of about 0.1 to about 5.0 poise.

2-6. (canceled)

7. The kit of claim 1, wherein the lotion contains about 0.005% to about 0.05% by weight of clobetasol propionate.

8. The kit of claim 1, wherein the metered-dose dispenser comprises a high density polyethylene (HDPE) bottle.

9. The kit of claim 1, wherein the metered dose dispenser comprises a bottle with a volume capacity of about 40 ml to 150 ml.

10. The kit of claim 1, wherein the metered-dose dispenser comprises an integral pump locking mechanism.

11. The kit of claim 1, wherein the metered-dose dispenser further comprises an actuator, and wherein any pharmaceutical composition that is not applied to the patient is drawn back into the actuator.

12. The kit of claim 11, wherein each actuation delivers about 0.15 mg±20% of Clobetasol propionate.

13. The kit of claim 12, wherein each actuation of the actuator delivers about 0.15 mg±20% of clobetasol propionate in about 0.3 gm±20% of lotion.

14. The kit of claim 1, wherein the metered-dose dispenser further comprises a cap.

15. The kit of claim 14, wherein the cap is used as an applicator.

16. The kit of claim 1, wherein a dose counter is attached to the metereddose dispenser.

17. A method of treating corticosteroid-responsive dermatoses comprising administering clobetasol propionate lotion twice daily to affected skin on a patient in need thereof from metered-dose dispenser, wherein total dosage administered to the patient does not exceed 50 gm of clobetasol propionate lotion per week.

18. The method of claim 17, wherein said administration of Clobetasol propionate lotion is limited to two consecutive weeks.

19. A method for treating moderate to severe plaque psoriasis comprising administering clobetasol propionate lotion twice daily to affected skin of patient in need thereof from a metered-dose dispenser, wherein total dosage does not exceed 50 gm of the clobetasol propionate lotion per week.

20. The method of claim 19, wherein the clobetasol propionate lotion is administered for the period of two consecutive weeks.

21. The method of claim 20, wherein the clobetasol propionate lotion is administered for up to an additional two weeks on localized lesions of less than 10% body surface area of the patient with moderate to severe plaque psoriasis.

22. (canceled)
